(19) United States
(12) Patent Application Publication
(10) Pub. No.: US 2008/0026980 A1
(43) Pub. Date: Jan. 31, 2008

(54) Use of Agents Derived from Ceacam1 for the Treatment of Inflammatory Diseases

(76) Inventors: Nalan Utku, Berlin (DE); Steven Richard Blumberg, Chestnut Hill,, MA (US)

Correspondence Address: COOPER & DUNHAM, LLP, 1185 AVENUE OF THE AMERICAS, NEW YORK, NY, 10036, US

(21) Appl. No.: 10/583,291

(22) PCT Filed: Dec. 17, 2004
(86) PCT No.: PCT/EP04/14435

§ 371 Date: Nov. 3, 2006

(30) Foreign Application Priority Data
Dec. 17, 2003 (EP) 03028968.0

(51) Int. Cl. A61K 38/02; A61P 19/02; C07K 16/00

(52) U.S. Cl. 514/2; 530/387.3

(57) ABSTRACT

The use of an agent that selectively modulates cross-linking of biliary glycoprotein polypeptides for the preparation of a pharmaceutical composition for preventing or treatment of a mammal subject afflicted with an inflammatory disease is provided. In particular, a method for preventing or treatment of a mammal subject afflicted with rheumatoid arthritis or multiple sclerosis, comprising the step of administering to a mammal in need thereof a therapeutic effective amount of a fusion protein of a fragment of biliary glycoprotein and a fragment of an immunoglobulin is described.

### FIELD OF THE INVENTION

The present invention relates to fusion protein derived from human biliary glycoprotein (CEACAM1), especially from the extracellular domain of CEACAM1, wherein said fusion protein is capable of modulating the function (e.g., signalling or adhesive activities) of CEACAM1 and/or its ligand. In particular, the fusion protein of the invention is capable of suppressing the proliferation of activated cells of the immune system. Furthermore, the present invention relates to compositions comprising said fusion protein and to methods of modulating immune cell proliferation, and treating immune response related diseases.

### BACKGROUND OF THE INVENTION

T-cell activation is a serial process involving multiple signaling pathways and sequential changes in gene expression resulting in differentiation of T-cells into distinct subpopulations, i.e. Th1 and Th2, which are distinguishable by their pattern of cytokine production and characterize the mode of cellular immune response. The T-cell response is initiated by the interaction of the antigen-specific T-cell receptor (TCR) with peptide presented by major histocompatibility complex (MHC) molecules on the surface of antigen presenting cells (APCs). Additional signals are provided by a network of receptor-ligand interactions mediated by a number of membrane proteins such as CD28/CTLA4 and B7, CD40/CD40L, LFA-1 and ICAM-1 (Lenschow, Science 257 (1992), 789-792; Linsley, Annu. Rev. Immunol. 11 (1993), 191-212; Xu, Immunity 1 (1994), 423-431; Bachmann, Immunity 7 (1997), 549-557; Schwartz, Cell 71 (1992), 1065-1068) collectively called costimulatory signals (Perez, Immunity 6 (1997), 411). These membrane proteins can alter T-cell activation in distinct ways (Bachmann, Immunity 7 (1997), 549-557) and regulate the immune response by the integration of positive and negative signals provided by these molecules (Bluestone, Immunity 2 (1995), 555-559; Perez, Immunity 6 (1997), 411). Many of the agents which are effective in modulating the cellular immune response either interfere with the T-cell receptor (Cosimi, Transplantation 32 (1981), 535-539) block costimulatory signaling (Larsen, Nature 381 (1996), 434-438; Blazar J. Immuno. 157 (1996), 3250-3259; Kirk, Proc. Natl. Acad. Sci. USA 94 (1997), 8789-8794; Linsley, Science 257 (1992), 792-95; Turka, Proc. Natl. Acad. Sci. USA 89 (1992), 11102-11105) or inhibit intracellular activation signals downstream from these primary cell membrane triggers (Schreiber and Crabtree, Immunology Today 13 (1992), 136-42). Therapeutic prevention of T-cell activation in organ transplantation and autoimmune diseases presently relies on panimmunosupressive drugs interfering with downstream intracellular events. Specific modulation of the immune response remains a longstanding goal in immunological research.

In view of the need of therapeutic means for the treatment of diseases related to immune responses of the human body, the technical problem of the present invention is to provide means and methods for modulation of the immune response in a subject. The solution to said technical problem is achieved by providing the embodiments characterized in the claims, and described further below.

### SUMMARY OF THE INVENTION

The present invention is directed to the use of an agent that selectively modulates cross-linking of biliary glycoprotein polypeptides for the preparation of a pharmaceutical composition for preventing or treatment of a mammal subject afflicted with an inflammatory disease. In particular, the present invention relates to a method for preventing or treatment of a mammal subject afflicted with rheumatoid arthritis or multiple sclerosis, comprising the step of administering to a mammal in need thereof a therapeutic effective amount of a fusion protein comprising at least a fragment of biliary glycoprotein and at least a fragment of an immunoglobulin or derivative thereof.

The use of the therapeutic agent in accordance with the present invention may be accompanied by the use of further therapeutic agents such as other inflammatory agents.

### BRIEF DESCRIPTION OF THE DRAWINGS

FIG. 1: Amino acid sequence of human CEACAM1 protein. The underlined amino acid sequences provides a preferred fragment to be utilized to generate the CEACAM1 fusion protein.

FIG. 2: CEACAM1 protein significantly reduced PBMC-proliferation after PHA-stimulation. CEACAM1 fusion protein (CEACAM1-) inhibits human PBMC proliferation.

FIG. 3: CEACAM1-fusions protein inhibits IFNgamma (IFNg)-production significantly in co-culture with PHA-stimulated PBMC. CEACAM1 fusion protein (CEACAM1-Fc) inhibits human PBMC interferon gamma expression.

FIG. 4: CEACAM1-fusion protein inhibits proliferation of mice splenocytes in co-culture with PHA and Interleukin 2. CEACAM1 fusion protein (CEACAM1-Fc) cross-reacts with mice CEACAM1 and inhibits the proliferation of PHA and IL-2 activated mice splenocytes.

### DESCRIPTION OF THE INVENTION

The present invention relates to the use of an agent that selectively modulates cross-linking of biliary glycoprotein polypeptides for the preparation of a pharmaceutical composition for preventing or treatment of a mammal subject afflicted with an inflammatory disease.

Without intending to be bound by theory, it is believed that fusion proteins derived from CEACAM1 and described herein are capable of modulating the function (e.g., signaling or adhesive activities) of CEACAM1, its family members and/or their ligands, for example by interfering with the interaction of CEACAM1 with its ligand.

The term “interfering with the interaction of CEACAM1 with its ligand” means in accordance with the present invention that said agent, e.g. fusion protein or analog or derivative thereof is capable of inhibiting and/or modulating the interaction of CEACAM1 with its corresponding ligand. Since the interaction of CEACAM1 with its ligand(s) modulates events which are valuable in course of immune responses such fusion proteins should also be capable of modulating immune responses. In accordance with the present invention said agent, e.g. fusion protein preferably interacts with the CEACAM1-ligand, for example by specifically binding to said ligand. The term “ligand” includes small molecules and soluble binding proteins as well a membrane associated receptors. “Specifically binding” means “specifically interacting with” whereby said interaction may be, inter alia, covalently, non-covalently and/or hydrophobic.

Biliary glycoprotein (BGP) is also known as CD66a, CEACAM1 and C-CAM1. Hence, those terms are used interchangeably herein. BGP is a member of the carcinoembryonic antigen family (CEA), is an inhibitory receptor for activated T cells contained within the human intestinal epithelium; see WO99/52552 and Morales et al. J. Immunol. 163 (1999), 1363-1370. Biliary glycoprotein binding agents and their use for generally modulating the immune response are described in WO99/52552.

As used herein, the term “mammal” means any member of the higher vertebrate animals included in the class Mammalia, as defined in Webster's Medical Desk Dictionary 407 (1986), and includes but is not limited to humans, other primates, pigs, dogs, and rodents (such as immune suppressed mice). In the preferred embodiment of this invention, the mammal is a human.

The term inflammatory disease includes rheumatoid arthritis (RA), multiple sclerosis (MS), inflammatory bowel disease (IBD), and allergic asthma. In a preferred embodiment, said inflammatory disease to be treated in accordance with the present invention is arthritis or multiple sclerosis (MS), most preferably said inflammatory disease is rheumatoid arthritis (RA).

In one embodiment, said agent is an antibody, preferably a monoclonal antibody. Therapeutic anti-BGP antibodies are described, for example, in international patent application PCT/US03/28416.

However, in an alternative and preferred embodiment the agent comprises a ligand for the biliary glycoprotein polypeptide, wherein the ligand binds one, preferably two or more biliary glycoprotein polypeptides.

In a preferred embodiment, the ligand is fused to an immunoglobulin molecule or a fragment thereof. Likewise preferred is that the ligand comprises a biliary glycoprotein polypeptide or fragment thereof.

BGP, and its mouse and rat homologues C-CAM (Rosenberg et al., Cancer Res. 53 (1993), 4938-4945; Lin et al., J. Biol. Chem. 264 (1989), 14408-14414; Obrink, BioEssays 13 (1991), 227-234), have been regarded mainly as molecules which function in cell-cell adhesion that are expressed primarily by epithelial cells of the gastrointestinal tract and biliary tree, neutrophils and, more recently, B cells. BGP also serves as a receptor for mouse hepatitis virus (Williams et al., Proc. Natl. Acad. Sci. 88 (1991), 5533-5536) and for Opa proteins of Neisseria species of bacteria (Virji et al., Mol. Microbiol. 5 (1996), 941-950). It is of interest that ligation of BGP on epithelial cells may deliver a negative growth signal which may be decreased during tumor formation due to diminished expression of BGP (Rosenberg et al., 1993; Kunath et al., Oncogene, 11 (1995), 2375-2382; Brumer et al., Oncogene, 11 (1995), 1649-1655).

Previous studies indicated that the expression of BGP on activated human iIELs and, potentially, other mucosal T lymphocytes is involved in the down-regulation of cytolytic function; see Morales et al., J. Immunol. 163 (1999), 1363-1370. Hence, compounds capable of modulating the cytolytic activity of intestinal intraepithelial lymphocytes (iIELs) could be envisaged, which exerts their modulatory function through BGP.

The present invention provides a novel and more focused application of BGP in the treatment of specific inflammatory disease, especially RA and MS.

As mentioned before, in a first aspect the present invention provides a CEACAM1 fusion protein capable of modulating the immune response. CEACAM1 proteins play a major role in immune response. Recent investigations revealed a possible reaction between CEACAM1 as homophilic interaction with itself. In order to elucidate this binding and its consequences, a human CEACAM1 fusion protein was produced and examined regarding the functional activity on human peripheral blood mononuclear cells (PBMCs) in vitro; see Example 1. In addition cytokine production was investigated to determine whether, like with CEACAM1 antibodies, Th1-cytokine production would be inhibited by co-cultivation with CEACAM1 fusion protein; see Example 2.

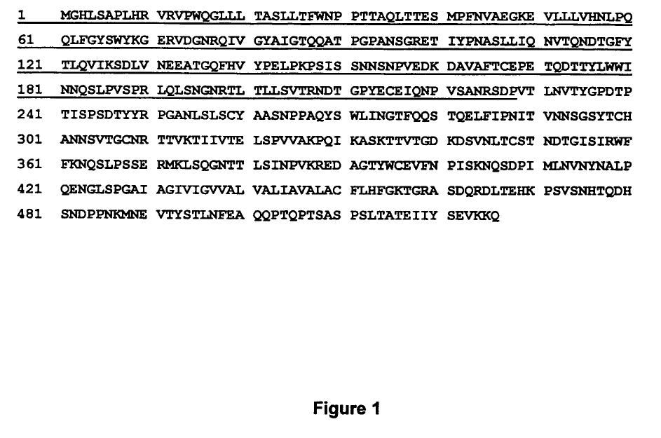

Thus, the present invention provides isolated fusion proteins derived CEACAM1 protein that include at least a fragment of the amino acid sequence of CEACAM1, e.g. that shown in FIG. 1 or analogs thereof that modulate the function of CEACAM1 protein and/or at least one ligand thereof.

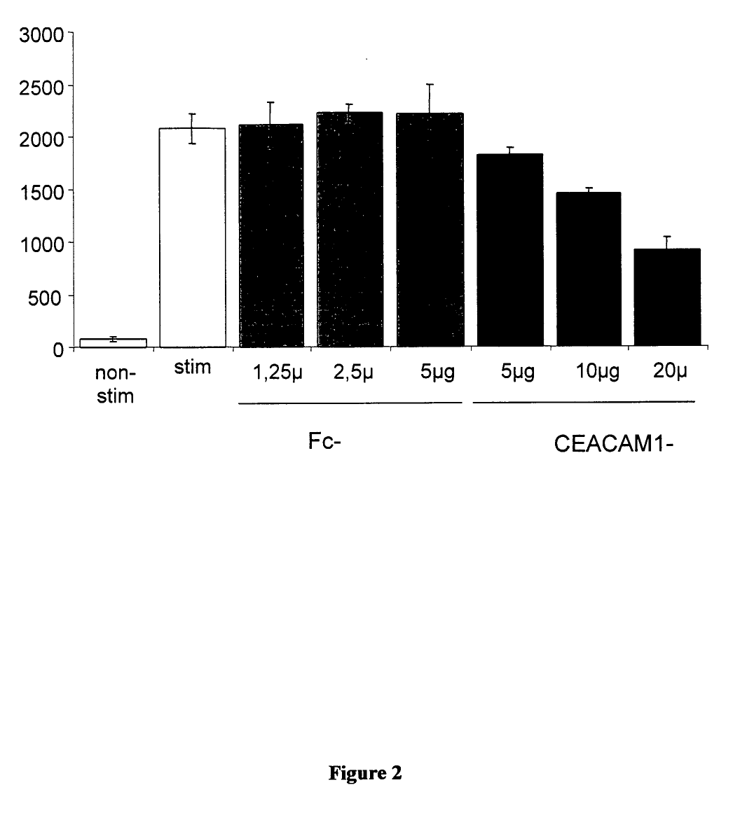

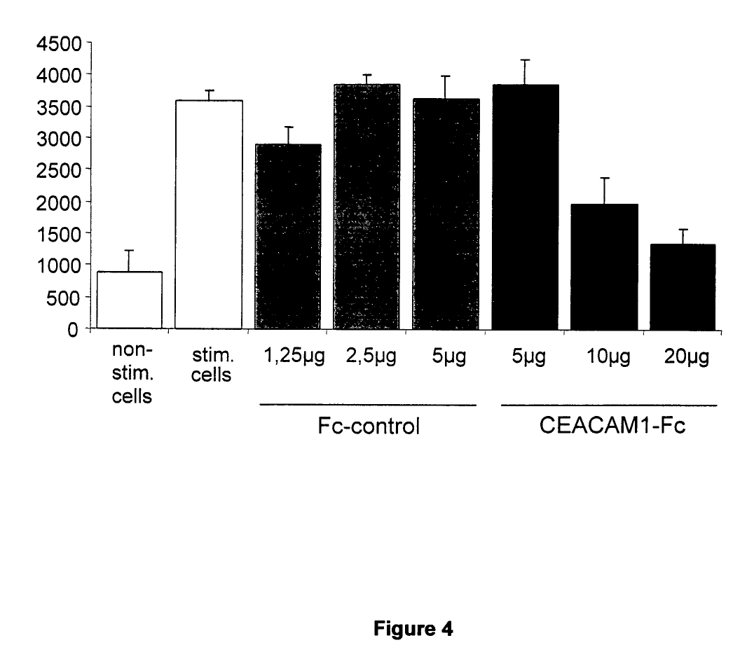

The fusion proteins were tested for their ability to inhibit proliferation of PHA-stimulated PBMCs and as shown in FIGS. 2 and 4 was found to be potent inhibitor of human PBMC and mouse splenocytes proliferation, respectively, while control Fc had not exhibited any activity. Accordingly, the fusion protein of the present invention is expected to be useful in the modulation of immune responses. Modulating the immune response, as for example by activating or inhibiting the proliferation and/or differentiation of T-cells, B-cells, NK cells, LAK cells, dendritic cells, monocytes, macrophages or other immune system cells, may be useful in treating autoimmune diseases, allergic diseases, and in transplantation therapies where graft vs. host or host vs. graft effects may be undesirable. The fusion protein could also be immune stimulants in settings such as cancer, infectious disease, sepsis, wound healing, or immunization. Alternatively, they could be immune suppressants. They could also be used to detect inflammation, and preferably modulate inflammation by activating or inhibiting activation of immune or inflammatory cells. A preferred method involves detecting (and preferably modulating) inflammation in tissues such as inflamed vessels tissue or leukocytes.

In one preferred embodiment the fusion protein or CEACAM1 peptide and peptide conjugates are used for inducing or maintaining immune unresponsiveness in a subject. The term “immune unresponsiveness” comprises non-unresponsiveness of immune cell subsets like T-cell or B-cells, NK-cells, monocytes and/or macrophages.

Thus, the present invention provides fusion protein derived CEACAM1 protein that are capable of modulating (i.e., altering by increasing, decreasing, etc.), for example, immune cell activation, cell proliferation, cell differentiation, or binding of CEACAM1 to its ligands. Preferably, the present invention provides isolated fusion protein comprising or consisting of an amino acid sequence indicated as CAECAM1 whole extracellular domain in FIG. 1, or a fragment thereof.

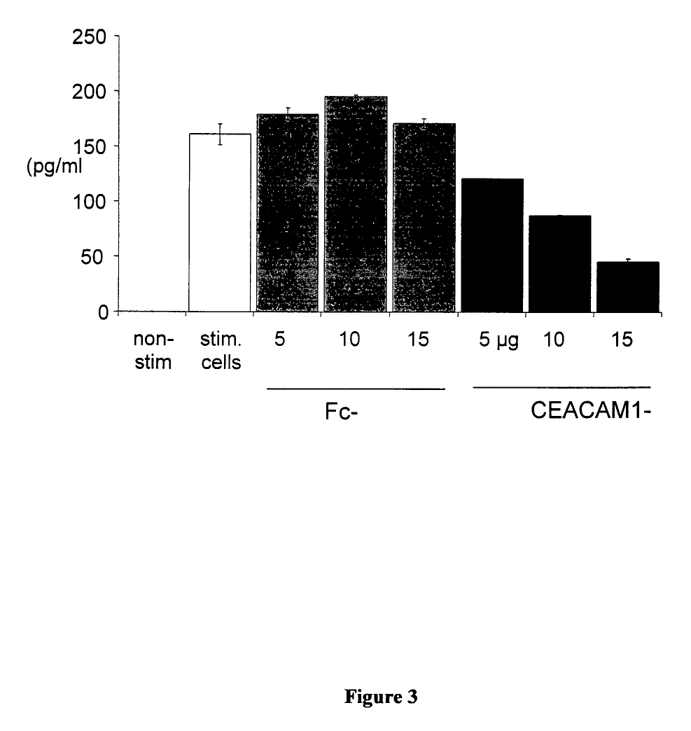

In addition, fusion proteins of the present invention have been shown in PHA-stimulated PMBCs to inhibit IFNγ-production (see FIG. 3). Accordingly, the fusion protein of the invention are preferably used for specific inhibition or modulation of IFN-γ dependent immune reactions which refers to Th1 type of immune responses associated with transplant rejection, multiple sclerosis, Type I diabetes mellitus or rheumatoid arthritis with or without a modulatory effect on Th2 immune reactions.

In this respect, experiments performed in accordance with the present invention demonstrated that the fusion protein of the present invention provides a short to medium term effectiveness in preventive CIA mouse model such as those described in Wang et al., Nature Immunology 2 (2001), 632-637. In particular, DBA/1 mice, having the disease induced by collagen s.c. (Sigma, 100 μg in cFA at day 0 and 100 μg in iFA at day 21), have been treated successfully with BGP-Fc fusion protein at a concentration of daily 300 μg i.p.

In addition, it could be shown that BGP-Fc fusion protein demonstrates therapeutic effectiveness in EAE mouse model of MS such as those described by Miller and Karpus (1996) Experimental autoimmune encephalomyelitis in the mouse. In: Coligan, Kruisbeek, Margulies, Shevach, and Strober (Ed.), Current Protocols in Immunology, 3 John Wiley & Sons Inc., USA, unit 15.1.10. In particular, SJL/J mice, having the disease induced by 150 μg PLP139-151 (Bachem) s.c. and 400 ng pertussis toxin (Sigma) i.p. at day 0 and 400 ng pertussis toxin (Sigma) i.p. at day 2, have been treated successfully with BGP-Fc fusion protein at a concentration of 500 μg i.p.

Thus, compositions comprising the fusion protein of this invention can be added to cells in culture (in vitro) or used to treat patients, such as mammals (in vivo). Where the fusion protein are used to treat a patient, the polypeptide is preferably combined in a pharmaceutical composition with a pharmaceutically acceptable carrier such as a larger molecule to promote polypeptide stability or a pharmaceutically acceptable buffer that serves as a carrier for the polypeptide or incorporated in a peptide conjugate that has more than one peptide coupled to a single entity.

The biological activity of the fusion protein identified here suggests that they have sufficient affinity to make them potential candidates for drug localization to cells expressing the appropriate surface structures. This targeting and binding to cells could be useful for the delivery of therapeutically active agents (including targeting drugs, DNA sequences, RNA sequences, lipids, proteins (e.g., human growth factors)) and gene therapy/gene delivery. More preferably, the therapeutically active agent is an, anti-inflammatory agent.

Molecules/particles with a specific number of amino acids of the fusion protein derived from CEACAM1 protein would bind specifically to cells/tissues expressing specific ligand combinations, and therefore could have diagnostic and therapeutic use. Thus, the fusion protein of the present invention can be labelled (e. g., fluorescent, radioactive, enzyme, nuclear magnetic) and used to detect specific targets in vivo or in vitro including “immunochemistry” like assays in vitro. In vivo they could be used in a manner similar to nuclear medicine imaging techniques to detect tissues, cells, or other material expressing CEACAM1 or its ligand.

The fusion protein of the present invention can be in their free acid form or they can be amidated at the C-terminal carboxylate group. The present invention also includes analogs of the polypeptide of, for example, those shown in FIG. 1, which typically have structural similarity with the corresponding amino acid sequences, preferably those of fusion protein CEACAM1.

The present invention also includes analogs of CEACAM1 fusion protein. An “analog” of a polypeptide includes at least a portion of the polypeptide, wherein the portion contains deletions or additions of one or more contiguous or non-contiguous amino acids, or containing one or more amino acid substitutions. Substitutes for an amino acid in the fusion protein of the invention are preferably conservative substitutions, which are selected from other members of the class to which the amino acid belongs. An analog can also be a larger peptide that incorporates the fusion protein described herein. For example, it is well-known in the art of protein biochemistry that an amino acid belonging to a grouping of amino acids having a particular size or characteristic (such as charge, hydrophobicity and hydrophilicity) can generally be substituted for another amino acid without substantially altering the structure of a polypeptide. For the purposes of this invention, conservative amino acid substitutions are defined to result from exchange of amino acids residues from within one of the following classes of residues: Class 1: Ala, Gly, Ser, Thr, and Pro; Class II: Cys, Ser, Thr, and Tyr; Class III: Glu, Asp, Asn, and Gln (carboxyl group containing side chains): Class IV: His, Arg, and Lys (representing basic side chains); Class V: Ile, Val, Leu, Phe, and Met (representing hydrophobic side chains); and Class VI: Phe, Trp, Tyr, and His (representing aromatic side chains). The classes also include other related amino acids such as halogenated tyrosines in Class VI.

Polypeptide analogs, as that term is used herein, also include modified fusion protein. Modifications of fusion proteins of the invention include chemical and/or enzymatic derivatizations at one or more constituent amino acid, including side chain modifications, backbone modifications, and N- and C-terminal modifications including acetylation, hydroxylation, methylation, amidation, and the attachment of carbohydrate or lipid moieties, cofactors, and the like.

In a preferred embodiment the peptide of the present invention comprises one of the group of D-isomer amino acids, L-isomer amino acids, or a combination thereof. The preparation of fusion protein comprising D-isomer amino acids is described for example in Schumacher, Science 271 (1996), 1854-1857.

A preferred polypeptide analog is characterized by having at least one of the biological activities described herein. Such an analog is referred to herein as a “biologically active analog” or simply “active analog”. The biological activity of a polypeptide can be determined, for example, as described in the Examples.

The fusion protein of the invention or part thereof may be synthesized by the solid phase method using standard methods based on either t-butyloxycarbonyl (BOC) or 9 fluorenylmethoxy-carbonyl (FMOC) protecting groups. This methodology is described by G. B. Fields et al. in Synthetic Fusion protein: A User's Guide, W. M. Freeman & Company, New York, N.Y., pp. 77-183 (1992). The present fusion protein may also be synthesized via recombinant techniques well known to those skilled in the art. For example, U.S. Pat. No. 5,595,887 describes methods of forming a variety of relatively small fusion protein through expression of a recombinant gene construct coding for a fusion protein which includes a binding protein and one or more copies of the desired target peptide. After expression, the fusion protein is isolated and cleaved using chemical and/or enzymatic methods to produce the desired target peptide.

In a particularly preferred embodiment, said biliary glycoprotein is a human biliary glycoprotein or a fragment thereof. In one embodiment the CEACAM1 protein or peptide conjugate of the present invention comprises at least a fragment of the amino acid sequence shown in FIG. 1. Active fusion proteins could be larger or smaller than the ones specifically described here. While the present fusion protein described are of about 233 amino acids, fusion proteins containing a number of amino acid residues, e.g., up to about 50 or 100 amino acid residues or more, that contain the described fusion protein, portions thereof, or similar fusion protein may have biological activity as well. Similarly, fusion proteins smaller than those shown in FIG. 1 may also have similar biological activity. Similarly, fusion proteins with amino acid substitutions or other alterations may block the activities of the described fusion protein or the parent molecules. Cyclic or otherwise modified forms of the fusion protein would also be expected to have biological activity. Preferably, the fusion protein of the present invention have about 233+/−5 to 10 amino acids derived from CEACAM1 protein.

Thus, the present invention provides isolated fusion proteins that include an amino acid sequence represented in FIG. 1 or analogs thereof that modulate the function of at least one CEACAM1 protein and/or at least one ligand thereof. These amino acid sequences can form a part of a larger peptide. Additionally, they can be used in various combinations in any one peptide. Preferably, the present invention provides isolated fusion protein indicated in FIG. 1 or analogs thereof. It is believed that these portions of certain of the fusion protein described herein contribute significantly to the activity of the fusion protein.

Similarly, fusion protein with amino acid substitutions or other alterations may block the activities of the described fusion protein or the parent molecules. Cyclic or otherwise modified forms of the fusion protein would also be expected to have biological activity.

The present fusion proteins are preferably capable of modulating, preferably inhibiting proliferation of peripheral blood mononuclear cells (PBMCs). Preferably, the fusion protein of the present invention modulate at least one of the following (which are functions of CEACAM1 proteins and/or ligands thereof): activation of neutrophils; activation or inhibition of T-cells, B-cells, NK cells, LAK cells, dendritic cells, or other immune system cells; proliferation and/or differentiation of T-cells, B-cells, NK cells, LAK cells, dendritic cells, or other immune system cells; proliferation and/or differentiation of epithelial cells such as breast or intestinal/colonic epithelium cells or keratinocytes. In addition these fusion protein are preferably capable of altering homotypic and/or heterotypic adhesion among CEACAM1 proteins (i.e., CEACAM1 family members) or adhesion of CEACAM1 proteins to other CEACAM1 ligands.

The present invention also provides peptide conjugates. The ability of fusion protein complexed with carrier molecules or structures, such as microbeads, liposomes, biological carrier molecules, synthetic polymers, biomaterials, and cells, thereby forming peptide conjugates is shown to impart the larger structure with the ability to bind to cells expressing CEACAM1 or its ligand. Such peptide conjugates bind to cells expressing a CEACAM1 protein or a CEACAM1 ligand.

The fusion protein or peptide conjugates of the present invention can also include molecules for labeling (i. e., labels such as fluorescence tags, magnetic resonance tags, radioactive tags, enzymatic tags). In this way, these can be used in diagnostic methods to detect specific targets in vivo or in vitro.

The present invention also provides a method of modulating (e.g., activating or inhibiting) immune cell (e. g., T-cells, B-cells, NK cells, LAK cells, or dendritic cells) activation, proliferation, and/or differentiation that includes contacting an immune cell with a peptide or peptide conjugate described above.

In addition, some fusion proteins differ from these fusion protein by one or several amino acids and could compete with these active fusion protein or the natural CEACAM1 protein or ligand thereof for certain biological activities.

The methods described herein can be carried out in vitro or in vivo. The fusion protein can be used alone or in various combinations as well as in peptide conjugates. They are used in amounts that provide the desired effect. These amounts can be readily determined by one of skill in the art.

In a further preferred embodiment, said immunoglobulin which a part of the ligand, e.g. fusion protein of the invention is a human immunoglobulin or a fragment thereof. Most preferably, said immunoglobulin fragment of the immunoglobulin is the Fc portion of the immunoglobulin or of immunoglobulin-like molecules. A preferred source for the Fc region is, for example, the low affinity immunoglobulin gamma Fc region receptor II-b precursor (Fc-gamma RII-b) (FcRII-b) (IgG Fc receptor II-b) (Fc-gamma-RIIb) (CD32 antigen) (CDw32), the amino acid sequence of which is available under swissprot accession no. P31994; see also Warmerdam et al., Int. Immunol. 5 (1993), 239-247; Kyogoku et al., Arthritis Rheum. 46 (2002), 1242-1254; Strausberg et al., Proc. Natl. Acad. Sci. U.S.A. 99 (2002), 16899-16903; Brooks et al., J. Exp. Med. 170 (1989), 1369-1385. Of course, other suitable sources for Fc portions may be used as well.

In a preferred embodiment said biliary glycoprotein fragment comprises the amino sequence from position 1 to 100, more preferably from 1 to 150 and most preferably from about 1 to about 228 of SEQ ID NO: 2 (FIG. 1) or a fragment thereof and/or the immunoglobulin fragment comprises the hinge-CH2-CH3 region of the Fc portion of the immunoglobulin.

The present invention also relates to a fusion protein as described above, in particular comprising a human biliary glycoprotein fragment substantially consisting of the amino sequence from position 1 to 228 of SEQ ID NO: 2 (FIG. 1) or a fragment thereof and an Fc portion of an human immunoglobulin.

In addition, the present invention relates to polynucleotides encoding the described fusion protein. The polynucleotide of the invention encoding the above described antibody may be, e.g., DNA, cDNA, RNA or synthetically produced DNA or RNA or a recombinantly produced chimeric nucleic acid molecule comprising any of those polynucleotides either alone or in combination. Preferably said polynucleotide is part of a vector. Such vectors may comprise further genes such as marker genes which allow for the selection of said vector in a suitable host cell and under suitable conditions.

Preferably, the polynucleotide of the invention is operatively linked to expression control sequences allowing expression in prokaryotic or eukaryotic cells. Expression of said polynucleotide comprises transcription of the polynucleotide into a translatable mRNA. Regulatory elements ensuring expression in eukaryotic cells, preferably mammalian cells, are well known to those skilled in the art. They usually comprise regulatory sequences ensuring initiation of transcription and optionally poly-A signals ensuring termination of transcription and stabilization of the transcript. Additional regulatory elements may include transcriptional as well as translational enhancers, and/or naturally associated or heterologous promoter regions.

Possible regulatory elements permitting expression in prokaryotic host cells comprise, e.g., the PL, lac, trp or tac promoter in E. coli, and examples for regulatory elements permitting expression in eukaryotic host cells are the AOX1 or GAL1 promoter in yeast or the CMV-, SV40-, RSV-promoter (Rous sarcoma virus), CMV-enhancer, SV40-enhancer or a globin intron in mammalian and other animal cells.

Beside elements which are responsible for the initiation of transcription such regulatory elements may also comprise transcription termination signals, such as the SV40-poly-A site or the tk-poly-A site, downstream of the polynucleotide. Furthermore, depending on the expression system used leader sequences capable of directing the polypeptide to a cellular compartment or secreting it into the medium may be added to the coding sequence of the polynucleotide of the invention and are well known in the art. The leader sequence(s) is (are) assembled in appropriate phase with translation, initiation and termination sequences, and preferably, a leader sequence capable of directing secretion of translated protein, or a portion thereof, into the periplasmic space or extracellular medium. Optionally, the heterologous sequence can encode a fusion protein including a C- or N-terminal identification peptide imparting desired characteristics, e.g., stabilization or simplified purification of expressed recombinant product. In this context, suitable expression vectors are known in the art such as Okayama-Berg cDNA expression vector pcDV1 (Pharmacia), pCDM8, pRc/CMV, pcDNA1, pcDNA3 (Invitrogen), or pSPORT1 (GIBCO BRL).

Preferably, the expression control sequences will be eukaryotic promoter systems in vectors capable of transforming or transfecting eukaryotic host cells, but control sequences for prokaryotic hosts may also be used. Once the vector has been incorporated into the appropriate host, the host is maintained under conditions suitable for high level expression of the nucleotide sequences, and, as desired, the collection and purification of the fusion protein may follow; see, Beychok, Cells of Immunoglobulin Synthesis, Academic Press, N.Y., (1979).

Furthermore, the present invention relates to vectors, particularly plasmids, cosmids, viruses and bacteriophages used conventionally in genetic engineering that comprise a polynucleotide of the invention. Preferably, said vector is an expression vector and/or a gene transfer or targeting vector. Expression vectors derived from viruses such as retroviruses, vaccinia virus, adeno-associated virus, herpes viruses, or bovine papilloma virus, may be used for delivery of the polynucleotides or vector of the invention into targeted cell population. Methods which are well known to those skilled in the art can be used to construct recombinant viral vectors; see, for example, the techniques described in Sambrook, Molecular Cloning A Laboratory Manual, Cold Spring Harbor Laboratory (1989) N.Y. and Ausubel, Current Protocols in Molecular Biology, Green Publishing Associates and Wiley Interscience, N.Y. (1994). Alternatively, the polynucleotides and vectors of the invention can be reconstituted into liposomes for delivery to target cells. The vectors containing the polynucleotides of the invention can be transferred into the host cell by well known methods, which vary depending on the type of cellular host. For example, calcium chloride transfection is commonly utilized for prokaryotic cells, whereas calcium phosphate treatment or electroporation may be used for other cellular hosts; see Sambrook, supra.

The present invention furthermore relates to host cells transformed with a polynucleotide or vector of the invention. Said host cell may be a prokaryotic or eukaryotic cell. The polynucleotide or vector of the invention which is present in the host cell may either be integrated into the genome of the host cell or it may be maintained extrachromosomally. The host cell can be any prokaryotic or eukaryotic cell, such as a bacterial, insect, fungal, plant, animal or human cell. Preferred fungal cells are, for example, those of the genus Saccharomyces, in particular those of the species S. cerevisiae. The term “prokaryotic” is meant to include all bacteria which can be transformed or transfected with a DNA or RNA molecules for the expression of a fusion protein of the invention. Prokaryotic hosts may include gram negative as well as gram positive bacteria such as, for example, E. coli, S. typhimurium, Serratia marcescens and Bacillus subtilis. The term “eukaryotic” is meant to include yeast, higher plant, insect and preferably mammalian cells, most preferably NSO and CHO cells. Depending upon the host employed in a recombinant production procedure, the fusion protein encoded by the polynucleotide of the present invention may be glycosylated or may be non-glycosylated. The fusion protein of the invention may also include an initial methionine amino acid residue. A polynucleotide of the invention can be used to transform or transfect the host using any of the techniques commonly known to those of ordinary skill in the art. Furthermore, methods for preparing fused, operably linked genes and expressing them in, e.g., mammalian cells and bacteria are well-known in the art (Sambrook, Molecular Cloning: A Laboratory Manual, Cold Spring Harbor Laboratory, Cold Spring Harbor, N.Y., 1989). The genetic constructs and methods described therein can be utilized for expression of the antibody of the invention or the corresponding immunoglobulin chains in eukaryotic or prokaryotic hosts. In general, expression vectors containing promoter sequences which facilitate the efficient transcription of the inserted polynucleotide are used in connection with the host. The expression vector typically contains an origin of replication, a promoter, and a terminator, as well as specific genes which are capable of providing phenotypic selection of the transformed cells. Suitable source cells for the DNA sequences and host cells for inmmunoglobulin expression and secretion can be obtained from a number of sources, such as the American Type Culture Collection (“Catalogue of Cell Lines and Hybridomas,” Fifth edition (1985) Rockville, Md., U.S.A., which is incorporated herein by reference).

The fusion protein of the present invention may be employed in a monovalent state (e.g., free polypeptide or polypeptide coupled to a carrier molecule or structure).

The fusion protein may also be employed as conjugates having more than one (same or different) peptides/proteins bound to a single carrier molecule. The carrier molecule or structure may be microbeads, liposomes, biological carrier molecule (e.g., a glycosaminoglycan, a proteoglycan, albumin, or the like), a synthetic polymer (e.g., a polyalkyleneglycol or a synthetic chromatography support), biomaterial (e.g., a material suitable for implantation into a mammal or for contact with biological fluids as in an extracorporeal device), or other cell. Typically, ovalbumin, human serum albumin, other proteins, polyethylene glycol, or the like are employed as the carrier. Such modifications may increase the apparent affinity and/or change the stability of a peptide. The number of peptide fragments associated with or bound to each carrier can vary. In addition, as mentioned above, the use of various mixtures and densities of the fusion protein described herein may allow the production of complexes that have specific binding patterns in terms of preferred ligands. The fusion protein can be conjugated to other fusion proteins using standard methods known to one of skill in the art. Conjugates can be separated from free proteins through the use of gel filtration column chromatography or other methods known in the art.

For instance, protein conjugates may be prepared by treating a mixture of fusion protein and carrier molecules (or structures) with a coupling agent, such as a carbodiimide. The coupling agent may activate a carboxyl group on either the peptide or the carrier molecule (or structure) so that the carboxyl group can react with a nucleophile (e.g. an amino or hydroxyl group) on the other member of the peptide conjugate, resulting in the covalent linkage of the peptide and the carrier molecule (or structure).

As another example, fusion protein may be coupled to biotin-labeled polyethylene glycol and then coupled to avidin containing compounds. In the case of fusion protein coupled to other entities, it should be understood that the designed activity may depend on which end of the peptide is coupled to the entity.

The present invention also provides a composition that includes one or more active agents (i.e., fusion protein) of the invention and one or more pharmaceutically acceptable carriers. One or more fusion proteins with demonstrated biological activity can be administered to a patient in an amount alone or together with other active agents and with a pharmaceutically acceptable buffer. The fusion proteins can be combined with a variety of physiological acceptable carriers for delivery to a patient including a variety of diluents or excipients known to those of ordinary skill in the art. For example, for parenteral administration, isotonic saline is preferred. For topical administration, a cream, including a carrier such as dimethylsulfoxide (DMSO), or other agents typically found in topical creams that do not block or inhibit activity of the peptide, can be used. Other suitable carriers include, but are not limited to alcohol, phosphate buffered saline, and other balanced salt solutions.

The formulations may be conveniently presented in unit dosage form and may be prepared by any of the methods well known in the art of pharmacy. Preferably, such methods include the step of bringing the active agent into association with a carrier that constitutes one or more accessory ingredients.

The methods of the invention include administering to a subject, preferably a mammal, and more preferably a human, the composition of the invention in an amount effective to produce the desired effect. The fusion protein can be administered as a single dose or in multiple doses. Useful dosages of the active agents can be determined by comparing their in vitro activity and the in vivo activity in animal models. Methods for extrapolation of effective dosages in mice, and other animals, to humans are known in the art.

Hence, the present invention also relates to a method for preventing or treatment of a mammal subject afflicted with rheumatoid arthritis or multiple sclerosis, comprising the step of administering to a mammal in need thereof therapeutic effective amount of a fusion protein of a fragment of biliary glycoprotein and a fragment of an immunoglobulin.

The terms “treatment”, “treating” and the like are used herein to generally mean obtaining a desired pharmacological and/or physiological effect. The effect may be prophylactic in terms of completely or partially preventing a disease or symptom thereof and/or may be therapeutic in terms of partially or completely curing a disease and/or adverse effect attributed to the disease. The term “treatment” as used herein covers any treatment of a disease in a mammal, particularly a human, and includes: (a) preventing the disease from occurring in a subject which may be predisposed to the disease but has not yet been diagnosed as having it; (b) inhibiting the disease, i.e. arresting its development; or (c) relieving the disease, i.e. causing regression of the disease.

Furthermore, the term “subject” as employed herein relates to animals in need of amelioration, treatment and/or prevention of immunological diseases as disclosed herein. Most preferably said subject is a human.

In a preferred embodiment, the pharmaceutical composition of the present invention comprises at least one second agent, preferably an agent which inhibits T-cell stimulation depending on the intended use. Such agents include, for example, molecules that are capable of blocking or mimicking receptor/ligand interaction or the like which leads to T-cell suppression.

Such agents comprise those blocking the activity of, e.g., costimulatory molecules, such as anti CEACAM1 antibodies, and TNF-α blocking agents, e.g. antibodies, integrins, Ig-superfamily molecules, selectins as well as drugs blocking chemokines and their respective receptor interactions, drugs blocking IL2/IL2-receptor interaction and other conventional immunosuppressive drugs such as IL-2R mAbs, IL-Toxins and IL-Muteins. Examples for costimulatory molecules and their ligands are described in the prior art, e.g., in Schwartz, Cell 71 (1992), 1065-1068. The interruption of the receptor/ligand interactions by using mAbs or soluble CTLA41g for the interaction between CD28 to the B7-2 and CTLA4 to B7-1 and B7-2 are described in Blazar, J. Immunol. 157 (1996), 3250-3259; Bluestone, Immunity 2 (1995), 555-559; Linsley, Science 257 (1992), 792-95. Examples for blocking the receptor/ligand interaction by using mAbs to CD40 or CD40L are reported by Burden, Nature 381 (1996), 434-435; Kirk, Proc. Natl. Acad. Sci. USA 94 (1997), 8789-8794. CD2 antigen and its ligand LFA-3 are described in Bagogui Li et al., review in Adhesion Molecules, Fusion proteins, Novel Fusion protein, and Monoclonal Antibodies, Recent Developments in Transplantation Medicine, Vol. II, 1995, Physicians & Scientists Publishing Co., Inc. and blocking of their interaction by using of mAbs (anti-Leu-5b, OKT11, T11) is reported in Brumberg, Transplantation 51 (1991) 219-225 or CD2.1gG1 fusion protein. The use of monoclonal Abs agains CD4 molecule is described in Cosimi, Surgery 108 (1990), 406-414. CD47 blockade by mAbs is described by Rheinhold, J. Exp. Med. 185 (1997), 1-11. Integrins and 1g-superfamily molecules include LFA-1 with its ligand ICAM-1, -2, -3, Mac-1 with its ligand ICAM-1, -3; ICAM-1 with its ligand LFA-1, Mac-1, CD43; ICAM-2 with its ligand LFA-1; ICAM-3 with its ligand LFA-1, Mac-1; VLA4 and VCAM-1 see, e.g., David, Adams, review in Adhesion Molecules, Fusion proteins, Novel Fusion protein, and Monoclonal Antibodies, Recent Developments in Transplantation Medicine, Vol. II, 1995, Physicians&Scientists Publishing Co., Inc.; Isobe, Science, 255 (1992), 1125-1127; Cosimi, J. Immunology 144 (1990), 4604-4612; Hynes, Cell 69 (1992), 11-25.

Furthermore selectively interfering agents with VLA-4 mAbs to the alpha4 integrin chain (CD49d) can be used, beta1 integrin chain (CD29), or an activation-induced neo-epitope of VLA-4 as well as soluble VLA-4 ligands such as soluble fibronectin or its relevant peptide (GPEILDVPST), or soluble VCAM-1 or its relevant peptide. More selectively blocking agents are antisense oligonucleotides, designed to selectively hybridize with cytoplasmic alpha4, beta1, or VCAM-1 mRNA; Fedoseyeva, J. Immunol. 57 (1994), 606-612.

Another example is the drug pentoxifylline (PTX) that is able to block expression of VCAM-1; Besler, J. Leukoc. Biol. 40 (1986), 747-754. Furthermore, VCAM-1 mAb, M/K-2, anti-murine, for example could prolong allograft survival, Orosz, Transplantation, 56 (1993), 453-460.

Blocking of members of the integrin family and their ligands by using mAbs is described in Kupiec-Weglinski, review in Adhesion Molecules, Fusion proteins, Novel Fusion protein, and Monoclonal Antibodies, Recent Developments in Transplantation Medicine, Vol. II, 1995, Physicians&Scientists Publishing Co., Inc.

Selectins, e.g., L-selectin (CD62L), E-selectin (CD62E), P-selectin (CD62P) have been described in Forrest and Paulson, Selectin family of adhesion molecules. In: Granger and Schmid-Schonbein, eds. Physiology and Pathophysiology of Leukocyte Adhesion. New York, Oxford Press, 1995, pp 68-146.

The combination of conventional immunosuppressive drugs, e.g., ATG, ALG, OKT3, Azathioprine, Mycophenylate, Mofetyl, Cyclosporin A, FK506, Sirolimus (Rapamune), Corticosteroids may be used as described in Cosimi, Transplantation 32 (1981), 535-539; Shield, Transplantation 38 (1984), 695-701, and Graft, June 2001, Vol 4 (4).

The interruption of chemokines and interactions with their respective receptor by using mAbs is reviewed in Luster, Chemokines-chemotactic cytokines that mediate inflammation, New Engl. J. Med. Feb. (1998), 436-445.

Thus, any agent as defined above and referenced by way of example can be used in accordance with the pharmaceutical composition of the invention or the methods and uses described herein.

The agents of the present invention are preferably formulated in pharmaceutical compositions and then, in accordance with the methods of the invention, administered to a patient, such as a human patient, in a variety of forms adapted to the chosen route of administration. The formulations include, but are not limited to, those suitable for oral, rectal, vaginal, topical, nasal, ophthalmic, or parental (including subcutaneous, intramuscular, intraperitoneal, intratumoral, intraorgan, intraarterial and intravenous) administration, or by inhalation.

Formulations suitable for parenteral administration conveniently include a sterile aqueous preparation of the active agent, or dispersions of sterile powders of the active agent, which are preferably isotonic with the blood of the recipient. Absorption of the active agents over a prolonged period can be achieved by including agents for delaying, for example, aluminum monostearate and gelatin.

Formulations of the present invention suitable for oral administration may be presented as discrete units such as tablets, troches, capsules, lozenges, wafers, or cachets, each containing a predetermined amount of the active agent as a powder or granules, as liposomes containing the active agent, or as a solution or suspension in an aqueous liquor or non-aqueous liquid such as a syrup, an elixir, an emulsion, or a draught. Such compositions and preparations typically contain at least about 0.1 wt-% of the active agent. The amount of polypeptide (i. e., active agent) is such that the dosage level will be effective to produce the desired result in the patient.

In accordance with the animal models used, the dosage can be in the range of 0.1 mg/kg/day to 25 mg/kg/day, preferably in the range of 0.1 mg to 10 mg.

Aerosol formulations such as nasal spray formulations include purified aqueous or other solutions of the active agent with preservative agents and isotonic agents. Such formulations are preferably adjusted to a pH and isotonic state compatible with the nasal mucous membranes. Formulations for rectal or vaginal administration may be presented as a suppository with a suitable carrier.

The appropriate concentration of the therapeutic agent might be dependent on the particular agent. The therapeutically effective dose has to be compared with the toxic concentrations; the clearance rate as well as the metabolic products play a role as do the solubility and the formulation. Therapeutic efficacy and toxicity of compounds can be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50.

These and other embodiments are disclosed and encompassed by the description and examples of the present invention. Further literature concerning any one of the materials, methods, uses and compounds to be employed in accordance with the present invention may be retrieved from public libraries and databases, using for example electronic devices. For example the public database “Medline” may be utilized, which is hosted by the National Center for Biotechnology Information and/or the National Library of Medicine at the National Institutes of Health. Further databases and web addresses, such as those of the European Bioinformatics Institute (EBI), which is part of the European Molecular Biology Laboratory (EMBL) are known to the person skilled in the art and can also be obtained using internet search engines. An overview of patent information in biotechnology and a survey of relevant sources of patent information useful for retrospective searching and for current awareness is given in Berks, TIBTECH 12 (1994), 352-364.

The above disclosure generally describes the present invention. Several documents are cited throughout the text of this specification. Full bibliographic citations may be found at the end of the specification immediately preceding the claims. The contents of all cited references (including literature references, issued patents, published patent applications as cited throughout this application and manufacturer's specifications, instructions, etc) are hereby expressly incorporated by reference; however, there is no admission that any document cited is indeed prior art as to the present invention.

The above disclosure generally describes the present invention. A more complete understanding can be obtained by reference to the following specific examples which are provided herein for purposes of illustration only and are not intended to limit the scope of the invention.

### EXAMPLES

The examples which follow further illustrate the invention, but should not be construed to limit the scope of the invention in any way. Detailed descriptions of conventional methods, such as those employed herein can be found in the cited literature; see also “The Merck Manual of Diagnosis and Therapy” Seventeenth Ed. ed by Beers and Berkow (Merck & Co., Inc. 2003).

The practice of the present invention will employ, unless otherwise indicated, conventional techniques of cell biology, cell culture, molecular biology, transgenic biology, microbiology, recombinant DNA, and immunology, which are within the skill of the art.

Methods in molecular genetics and genetic engineering are described generally in the current editions of Molecular Cloning: A Laboratory Manual, (Sambrook et al., (1989) Molecular Cloning: A Laboratory Manual, 2nd ed., Cold Spring Harbor Laboratory Press); DNA Cloning, Volumes I and II (Glover ed., 1985); Oligonucleotide Synthesis (Gait ed., 1984); Nucleic Acid Hybridization (Hames and Higgins eds. 1984); Transcription And Translation (Hames and Higgins eds. 1984); Culture Of Animal Cells (Freshney and Alan, Liss, Inc., 1987); Gene Transfer Vectors for Mammalian Cells (Miller and Calos, eds.); Current Protocols in Molecular Biology and Short Protocols in Molecular Biology, 3rd Edition (Ausubel et al., eds.); and Recombinant DNA Methodology (Wu, ed., Academic Press). Gene Transfer Vectors For Mammalian Cells (Miller and Calos, eds., 1987, Cold Spring Harbor Laboratory); Methods In Enzymology, Vols. 154 and 155 (Wu et al., eds.); Immobilized Cells And Enzymes (IRL Press, 1986); Perbal, A Practical Guide To Molecular Cloning (1984); the treatise, Methods In Enzymology (Academic Press, Inc., N.Y.); Immunochemical Methods In Cell And Molecular Biology (Mayer and Walker, eds., Academic Press, London, 1987); Handbook Of Experimental Immunology, Volumes I-IV (Weir and Blackwell, eds., 1986). Reagents, cloning vectors, and kits for genetic manipulation referred to in this disclosure are available from commercial vendors such as BioRad, Stratagene, Invitrogen, and Clontech. General techniques in cell culture and media collection are outlined in Large Scale Mammalian Cell Culture (Hu et al., Curr. Opin. Biotechnol. 8 (1997), 148); Serum-free Media (Kitano, Biotechnology 17 (1991), 73); Large Scale Mammalian Cell Culture (Curr. Opin. Biotechnol. 2 (1991), 375); and Suspension Culture of Mammalian Cells (Birch et al., Bioprocess Technol. 19 (1990), 251); Extracting information from cDNA arrays, Herzel et al., CHAOS 11, (2001), 98-107.

### Example 1

### Cell Proliferation

PBMC of healthy donors were isolated according to the Ficoll-Paque density centrifugation protocol. Samples of 50000 PBMC's/well were stimulated with PHA (1 μg/ml) and incubated for 48 h at 5% CO₂, 37° C. in presence of CEACAM1-fusion protein and ) and Fc-control in equimolar ratio (4:1) in a total volume of 100 μl/well. Samples were run in triplicates on 96 well-microtiter-plates (MTP's). After 48 h 0.5 μCi ³H-thymidine per well were added and the cells were re-incubated for additional 18 h. Cells were harvested and scintillation counting was performed using a beta counter. As shown in FIG. 2 CEACAM1-fusion protein significantly inhibited proliferation of PHA-stimulated PBMC in vitro.

### Example 2

### Quantitation of Secreted Cytokines in the Supernatant

PBMC of healthy donors were isolated according to the Ficoll-Paque density centrifugation protocol. Samples of 50000 PBMC's/well were stimulated with PHA (1 μg/ml) and incubated for 48 h at 5% CO₂, 37° C. in presence of HLA-fusion protein and controls (BSA) in a total volume of 100 μl/well. Samples were run in triplicates on 96 well-microtiter-plates (MTP's). After 48 h MTP's were centrifuged at 300×g for 10 min and supernatants collected from the wells. The quantitation of Interferon-γ (IFNg) in the supernatant was carried out on anti-IFNg-antibody-coated microtiter strips provided with the Cytoscreen® ELISA Kit, Biosource. The formerly collected supernatants (diluted 1:2 in dilution buffer) and diluted standards were incubated in presence of a biotinylated secondary antibody recognizing IFNg for 1.5 h at room temperature on these strips. Afterwards excessive secondary antibody was removed by washing 3 times with washing buffer. A streptavidin-peroxidase conjugate was added and incubated for 45 min at room temperature. Excessive conjugate was removed by washing. TMB-substrate-solution was added and the strips incubated for additional 30 min in the dark followed by the addition of stop solution. The colour development was measured at 450 nm and the numbers were statistically analyzed.

As shown in FIG. 3 CEACAM1 fusion protein significantly inhibited IFNg-production whereas an reduction up to 60% was observed of PHA-stimulated PBMC in vitro. In contrast there was no effect observed with control Fc.

Based on these prior shown experiments and the results presented in accordance with the present invention, one might hypothesize that the Th1-like cytokine IFNγ-production and the proliferation inhibiting effect of CEACAM1 derived-fusion protein are mediated through the signalling of CEACAM1 and homophilic interactions.

### Example 3

### Inhibition of Proliferation of Splenocytes Derived from Mice Proliferation

Lymphocytes were isolated from spleen using Medimachine. Cells were washed with PBS, centrifuged for 10 min, 1100 rpm at 21° C. and re-suspended in RPMI 1640 medium supplemented with 10% FCS, 2 mM L-glutamine and 100 U/ml streptomycin-penicillin. Number of cells was determined with Neubauer hemocytometer. Splenocytes were stimulated with PHA (10 μg/ml) and mIL-2 (100 U/ml) and incubated for 48 h at 5% CO₂, 37° C. in presence of BGP-Fc and Fc-control in equimolar ratio (4:1). After 48 h 0.5 μCi ³H-thymidine per well were added and the cells were re-incubated for additional 18 h. Cells were harvested and scintillation counting was performed using a beta counter.

As shown in FIG. 4, CEACAM1-fusion protein inhibited proliferation of PHA+IL-2-stimulated mice splenocytes significantly in a dose dependent manner whereas the control peptide showed no effect on proliferation. These results demonstrate that CEACAM1 ligation exhibits an antiproliferative effect via cross reacting with mice CEACAM1 in a similar manner as shown in FIG. 2 for human PBMC.

The complete disclosure of all patents, patent documents, and publications cited herein are incorporated by reference. The foregoing detailed description and examples have been given for clarity of understanding only. No unnecessary limitations are to be understood there from. The invention is not limited to the exact details shown and described, for variations obvious to one skilled in the art will be included within the invention defined by the claims.

1-15. (canceled)

16. A fusion protein comprising a human biliary glycoprotein (CEACAM1) fragment which is derived from the extracellular domain of CEACAM1 and an Fc portion of a human immunoglobulin.

17. The fusion protein of claim 16, wherein said CEACAM1 fragment substantially consists of the amino sequence from position 1 to 228 of SEQ ID NO: 2 (FIG. 1) or a fragment thereof.

18-20. (canceled)

21. A composition comprising the fusion protein of claim 16 optionally in combination with a pharmaceutically acceptable carrier.

22. A method for preventing or treatment of a mammal subject afflicted with rheumatoid arthritis or multiple sclerosis, comprising the step of administering to a mammal in need thereof a therapeutic effective amount of a fusion protein of a fragment of biliary glycoprotein and a fragment of an immunoglobulin.

23. The fusion protein of claim 16 comprising the hinge-CH2-CH3 region of the Fc portion of the immunoglobulin.

24. The method of claim 22, wherein said fusion protein is a fusion protein comprising a human biliary glycoprotein (CEACAM1) fragment which is derived from the extracellular domain of CEACAM1 and an Fc portion of a human immunoglobulin.

25. The method of claim 22, wherein the fusion protein is administered at a dosage in the range of 0.1 mg/kg/day to 25 mg/kg/day.

26. The method of claim 22, wherein the fusion protein is adapted in a form to be administered intravenously, subcutaneous, intramuscular or by inhalation.

27. A composition comprising the fusion protein of claim 17, optionally in combination with a pharmaceutically acceptable carrier.

28. The method of claim 22, wherein said fusion protein is a fusion protein comprising a human biliary glycoprotein (CEACAM1) fragment which is derived from the extracellular domain of CEACAM1 and an Fc portion of a human immunoglobulin, wherein said CEACAM1 fragment substantially consists of the amino sequence from position 1 to 228 of SEQ ID NO: 2 (FIG. 1) or a fragment thereof.

29. The method of claim 22, wherein said fusion protein is a fusion protein comprising a human biliary glycoprotein (CEACAM1) fragment which is derived from the extracellular domain of CEACAM1 and an Fc portion of a human immunoglobulin, and comprising the hinge-CH2-CH3 region of the Fc portion of the immunoglobulin.


SEQUENCE LISTING (1 sequence)

SEQ ID NO: 1 - protein, 526 aa, Homo sapiens
  CEACAM1 (1)..(526)
MGHLSAPLHRVRVPWQGLLLTASLLTFWNPPTTAQLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPSISSNNSNPVEDKDAVAFTCEPETQDTTYLWWINNQSLPVSPRLQLSNGNRTLTLLSVTRNDTGPYECEIQNPVSANRSDPVTLNVTYGPDTPTISPSDTYYRPGANLSLSCYAASNPPAQYSWLINGTFQQSTQELFIPNITVNNSGSYTCHANNSVTGCNRTTVKTIIVTELSPVVAKPQIKASKTTVTGDKDSVNLTCSTNDTGISIRWFFKNQSLPSSERMKLSQGNTTLSINPVKREDAGTYWCEVFNPISKNQSDPIMLNVNYNALPQENGLSPGAIAGIVIGVVALVALIAVALACFLHFGKTGRASDQRDLTEHKPSVSNHTQDHSNDPPNKMNEVTYSTLNFEAQQPTQPTSASPSLTATEIIYSEVKKQ